(19) United States
(12) Patent Application Publication
(10) Pub. No.: US 2007/0049892 A1
(43) Pub. Date: Mar. 1, 2007

(54) Absorbent article with core wrap

(75) Inventors: Patrick Robert Lord, Appleton, WI (US); Anthony John Wisneski, Kimberly, WI (US); Michael Barth Venturino, Appleton, WI (US); James George Van Himbergen, Kimberly, WI (US); Kambiz Bayat Makoui, Neenah, WI (US); Paul Eugene Olmstead, Menasha, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/216,587

(22) Filed: Aug. 30, 2005

(51) Int. Cl. A61F 13/15

(52) U.S. Cl. 604/385.16; 604/378

(57) ABSTRACT

An absorbent article includes an outercover, a bodyside liner, and an absorbent assembly positioned between the outercover and the bodyside liner. The absorbent assembly includes an absorbent core at least partially enveloped by at least one nonwoven core wrap. The absorbent assembly has a rear extension region length of at least 30 mm. The absorbent core may include at least 60 percent superabsorbent and the bodyside liner may include apertures.

### BACKGROUND OF THE INVENTION

Conventional absorbent articles have included an absorbent core which is composed of wood pulp fluff sandwiched between and bonded to an outercover layer and a liquid permeable facing sheet layer. The absorbent cores have also included particles of superabsorbent material. In addition, the absorbent structures have included one or more layers of tissue wrap material.

Conventional tissue wrap arrangements for absorbent cores have, however, not provided an adequate seal about the ends and sides of the absorbent core when the absorbent core includes relatively large amounts of superabsorbent material. As a result, excessive amounts of superabsorbent particles may migrate from the absorbent core and move to undesired locations within the absorbent article. The superabsorbent may migrate through the tissue wraps and/or out the sides and ends of the absorbent core. If the superabsorbent material moves to a location against the outercover, the dry particles may perforate the outercover and may escape the article. If the superabsorbent material moves to the bodyside liner, the wetted superabsorbent may produce an undesired gel against the wearer's skin. As a result, there remains a need for a product having improved containment of the absorbent core materials and a method and apparatus for producing the product.

### SUMMARY OF THE INVENTION

In response to these needs, an absorbent article includes an outercover, a bodyside liner and an absorbent assembly positioned between the outercover and the bodyside liner. The absorbent assembly includes a nonwoven core wrap at least partially enveloping an absorbent core. The absorbent assembly has a rear extension region length of at least 30 mm.

In some embodiments, the absorbent assembly may have a core wrap region length that is less than 90 percent of an absorbent assembly length. In some embodiments, the absorbent assembly may have a rear extension region length to a front extension region length of at least 1.5 to 1 or at least 2 to 1.

In some embodiments, the nonwoven core wrap may include thermoplastic fibers. In some embodiments, the nonwoven core wrap may be folded around the absorbent core and overlapped onto itself.

In some embodiments, the absorbent assembly has a core wrap region, a front extension region and a rear extension region. The nonwoven core wrap may be at least partially bonded to itself in the core wrap region and/or the front extension region and/or the rear extension region.

In some embodiments, the absorbent core may include at least 60 percent superabsorbent by weight. In some embodiments, the absorbent core has a liner facing surface and an outercover facing surface and the nonwoven core wrap may overlie the liner facing surface and partially overlie the outercover facing surface. The nonwoven core wrap may be adhesively joined with the outercover in the front extension region and/or the rear extension region and/or the core wrap region.

In another aspect, an absorbent article includes an outercover, a bodyside liner and an absorbent assembly positioned between the outercover and the bodyside liner. The absorbent assembly includes an absorbent core positioned between a first nonwoven core wrap and a second nonwoven core wrap. The absorbent assembly has a rear extension region length of at least 30 mm.

In various embodiments, the absorbent core may include at least 60 percent superabsorbent material by weight. In various embodiments, the bodyside liner may include a plurality of apertures. In various embodiments, the first nonwoven core wrap and the second nonwoven core wrap may include thermoplastic fibers.

In various embodiments, the first and the second nonwoven core wraps are at least partially bonded together in at least a front extension region and a rear extension region. In various embodiments, the ratio of the rear extension region length to a front extension region length is at least 2 to 1.

In various embodiments, the first nonwoven core wrap may be hydrophilic and the second nonwoven core wrap may be hydrophobic.

In various embodiments, the absorbent assembly has a front extension region length of at least 10 mm.

In another aspect, an absorbent article includes an outercover, a bodyside liner and an absorbent assembly positioned between the outercover and the bodyside liner. The absorbent assembly includes an absorbent core positioned between a first thermoplastic hydrophilic nonwoven core wrap and a second thermoplastic hydrophobic nonwoven core wrap. The absorbent core includes at least 60 percent superabsorbent and the absorbent assembly has a rear extension region length of at least 30 mm.

In various embodiments, the absorbent core has a liner facing surface, an outercover facing surface and lateral side edges. The first nonwoven core wrap overlies the liner facing surface, the lateral side edges and at least a portion of the outercover facing surface, the second nonwoven core wrap at least partially overlies the outercover facing surface, the first nonwoven core wrap being at least partially adhesively bonded to the second nonwoven core wrap in a front extension region and a rear extension region.

### BRIEF DESCRIPTION OF THE DRAWINGS

FIG. 1 representatively illustrates a partially cut-away, top plan view of a representative absorbent article of the present invention in a stretched and laid flat condition with the surface that contacts the wearer facing the viewer.

FIG. 2 representatively illustrates a partially cut-away, top plan view of an exemplary absorbent assembly of the present invention.

FIGS. 3-10 representatively illustrate partially exploded cross sectional views of exemplary absorbent articles of the present invention.

FIG. 11 representatively illustrates a schematic, side elevation view of an exemplary method and apparatus of the present invention.

FIG. 12 representatively illustrates a continuation of the schematic, side elevation view of FIG. 11.

FIG. 13 representatively illustrates an exemplary composite web formed during an intermediate step in the method of the present invention with portions cut away to illustrate underlying features.

FIG. 14 representatively illustrates a side perspective view of an exemplary forming drum of the present invention.

### DETAILED DESCRIPTION OF THE DRAWINGS

The absorbent article of the present invention will be described in terms of a diaper adapted to be worn by infants about the lower torso. It is understood that the absorbent article of the present invention is equally applicable to other articles such as adult incontinent products, training pants, feminine care products, and the like.

As used herein, the term “join”, and derivatives thereof, encompasses configurations wherein an element is directly secured to another element by affixing the element directly to the other element and configurations wherein the element is indirectly secured to the other element by affixing the element to intermediate member(s) which in turn are affixed to the other element.

As used herein, the term “nonwoven web” or nonwoven material refers to a fibrous web or material having a structure of individual fibers or filaments that are interlaid in a random pattern. Nonwoven webs may be formed, for example, by meltblowing, spunbonding, airlaying, wetlaying, drylaying, dry staple and carded web processes.

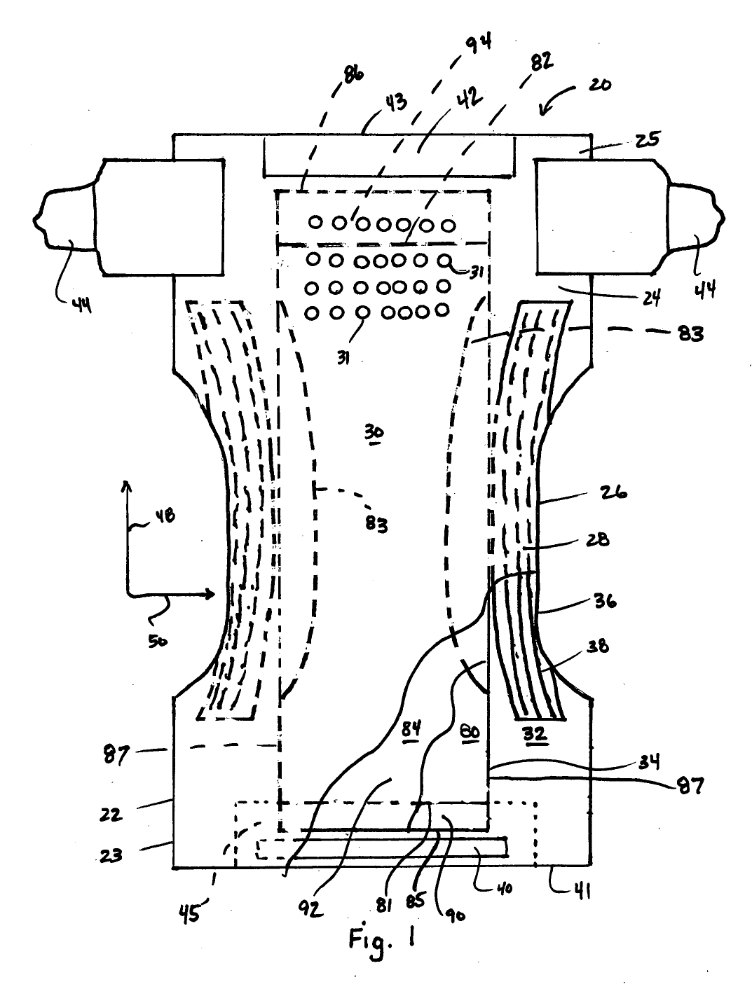

FIG. 1 representatively illustrates an embodiment of an absorbent article 20 of the present invention. The surface of the article which contacts the wearer is facing the viewer. The absorbent article 20 defines a front portion 22, a rear portion 24 and a crotch portion 26 connecting the front portion 22 and the rear portion 24. The front portion 22 defines a front waist region 23 and includes a front waist edge 41. The rear portion 24 defines a rear waist region 25 and includes a rear waist edge 43. The absorbent article 20 also defines a longitudinal direction 48 and a lateral direction 50. The absorbent article 20 includes a bodyside liner 30, an outercover 32 and an absorbent assembly 34 located between the bodyside liner 30 and the outercover 32. In various embodiments, the bodyside liner 30 may include one or more apertures 31.

The absorbent assembly 34 includes an absorbent core 80 and at least one core wrap 84. The absorbent core 80 has a front edge 81 and a rear edge 82. The front edge 81 and the rear edge 82 may be generally parallel and are opposed in the longitudinal direction 48. The absorbent core 80 also has laterally opposed side edges 83. The core wrap 84 has a front edge 85 and a rear edge 86. The front edge 85 and the rear edge 86 may be generally parallel and are opposed in the longitudinal direction 48. The core wrap 84 also has laterally opposed side edges 87.

The region between the core wrap front edge 85 and the absorbent core front edge 81 defines a front extension region 90. The region between the absorbent core front edge 81 and the absorbent core rear edge 82 defines a core wrap region 92. The region between the absorbent core rear edge 82 and the core wrap rear edge 86 defines a rear extension region 94.

As used herein, reference to a front portion refers to that part of the absorbent article which is generally located on the front of a wearer when in use. Reference to a front waist region refers to that part of the front portion which is located generally near the waist opening. Reference to the rear portion refers to the portion of the article generally located at the rear of the wearer when in use. Reference to a rear waist region refers to that part of the rear portion which is located generally near the waist opening. Reference to the crotch portion refers to that portion which is generally located between the legs of the wearer when in use.

The crotch portion 26 has opposite longitudinal side portions 28 which include a pair of elasticized, longitudinally-extending leg cuffs 36. The leg cuffs 36 are generally adapted to fit about the legs of a wearer in use and serve as a mechanical barrier to the lateral flow of body exudates. The leg cuffs 36 are elasticized by leg elastics 38. The absorbent article 20 may further include a front waist elastic 40 and/or a rear waist elastic 42. The rear portion 24 of the absorbent article 20 may further include a fastening means 44 which is adapted to hold the absorbent article 20 about the waist of the wearer when in use. The absorbent article 20 may also include a pair of containment flaps which extend longitudinally along the absorbent article 20 and are also adapted to provide a barrier to the flow of body exudates. It should be recognized that individual components of the absorbent article 20, such as the elastic members, may be optional depending upon the intended use of the absorbent article 20.

As used herein, the term “elastic” and derivatives thereof refers to materials or components that are generally capable of recovering their shape after deformation when the deforming force is removed. Specifically, as used herein, the term elastic or elastomeric is meant to be that property of any material or component which, upon application of a biasing force, permits that material or component to be stretchable to a stretched, biased length, which is at least about 125 percent, that is 1.25 times, its relaxed, unbiased length, and that will cause the material to recover at least 40 percent of its elongation upon release of the stretching, elongating force.

The bodyside liner 30 of the absorbent article 20 suitably presents a bodyfacing surface which is intended to be worn adjacent the body of the wearer and is compliant, soft feeling and nonirritating to the wearer's skin. Further, the bodyside liner 30 may be less hydrophilic than the absorbent assembly 34, to present a relatively dry surface to the wearer, and may be sufficiently porous to be liquid permeable, permitting liquid to readily penetrate through its thickness. A suitable bodyside liner 30 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 30 is suitably employed to help isolate the wearer's skin from fluids held in the absorbent assembly 34.

Various woven and nonwoven fabrics can be used for the bodyside liner 30. For example, the bodyside liner may be composed of a meltblown or spunbonded web of polyolefin fibers. The bodyside liner may also be a bonded-carded web composed of natural and/or synthetic fibers. The bodyside liner may be composed of a substantially hydrophobic material, and the hydrophobic material may, optionally, be treated with a surfactant or otherwise processed to impart a desired level of wettability and hydrophilicity. The bodyside liner may have one or more apertures 31 extending partially or completely through the thickness of the liner. In some embodiments, the bodyside liner may have a plurality of apertures 31 adapted to receive urine and/or fecal material, as is known in the art.

The outercover 32 of the absorbent article 20 may suitably be composed of a material which is either liquid permeable or liquid impermeable. It is generally preferred that the outercover 32 be formed from a material which is substantially impermeable to fluids. For example, a typical outercover can be manufactured from a thin plastic film or other flexible liquid-impermeable material. For example, the outercover 32 may be formed from a polyethylene film. If it is desired to present the outercover 32 with a more clothlike feeling, the outercover 32 may comprise a polyethylene film having a nonwoven web laminated to the outer surface thereof, such as a spunbond web of polyolefin fibers. Methods of forming such clothlike outercovers are known to those skilled in the art.

Further, the outercover 32 may be formed of a woven or nonwoven fibrous web layer which has been totally or partially constructed or treated to impart a desired level of liquid impermeability to selected regions that are adjacent or proximate the absorbent assembly 34. Still further, the outercover 32 may optionally be composed of a micro-porous “breathable” material which permits vapors to escape from the composite absorbent assembly 34 while still preventing liquid exudates from passing through the outercover 32.

The bodyside liner 30 and outercover 32 are generally joined to one another so as to form a pocket in which the absorbent assembly 34 is located. The bodyside liner 30 and outercover 32 may be joined directly to each other around the outer periphery of the absorbent article 20 by any means known to those skilled in the art, such as, for example, adhesive bonds, sonic bonds, thermal bonds, pressure bonds, and the like, and combinations thereof. For example, a uniform continuous layer of adhesive, a patterned layer of adhesive, a sprayed or meltblown pattern of adhesive or an array of lines, swirls or spots of adhesive may be used to join the bodyside liner 30 to the outercover 32. In some embodiments, the outercover 32 may include a full web spray of adhesive covering essentially the entire outercover 32. The full web spray may be adapted to join the outercover 32 with both the bodyside liner 30 and/or the core wrap 84 and/or the absorbent core 80.

Such bonding means may also be suitable for joining other components of the absorbent assembly and absorbent article of the present invention together. The leg cuffs 36 are suitably formed by portions of the outercover 32 and/or bodyside liner 30, which extend beyond the longitudinal sides of the composite absorbent assembly 34. Naturally, the leg cuffs 36 may also be formed from separate materials which are joined with the outercover 32 and/or bodyside liner 30.

The leg cuffs 36 may include leg elastics 38. Waist elastics 40 and 42 may also be provided. The leg elastics 38 are arranged to draw and hold the absorbent article 20 against the legs of the wearer. The waist elastics 40 and 42 are also arranged to draw and hold the absorbent article 20 against the wearer. In some embodiments, the absorbent article 20 may include a rear waist elastic 42 extending substantially the full width of the article. Materials suitable for use in forming leg elastics 38 and waist elastics 40 and 42 are known to those skilled in the art. Exemplary of such materials are strands or ribbons of a polymeric, elastomeric material which are adhered to the absorbent article 20 in a stretched position, or which are attached to the absorbent article while the article is pleated, such that elastic constrictive forces are imparted to the absorbent article 20. In a particular aspect of the invention, the elastics may be composed of individual strands of LYCRA which are available from INVISTA Co., a business having offices in Wichita, Kans., USA.

The leg elastics 38 and waist elastics 40 and 42 may have any configuration which provides the desired performance. For example, the leg elastics 38 and waist elastics 40 and 42 may comprise a single strand of elastic material, or may comprise several parallel or non-parallel strands of elastic material. The leg elastics 38 may be generally straight or optionally curved to more closely fit the contours of the legs and buttocks of the wearer and better contain bodily exudates. The leg elastics 38 and waist elastics 40 and 42 may be joined with the absorbent article 20 in any of several ways which are well known to those skilled in the art. For example, the elastics may be ultrasonically bonded, thermally bonded, pressure bonded, adhesively bonded, or the like, or combinations thereof to the absorbent article 20.

The fastening means 44 are typically joined to the corners of the rear portion 24 of the absorbent article 20 to provide a means for holding the article 20 on the wearer. Suitable fastening means 44 are well known to those skilled in the art and can include tape tab fasteners, hook and loop fasteners, mushroom and loop fasteners, snaps, pins, belts, and the like, and combinations thereof. Typically, the fastening means 44 are configured to be refastenable. It should also be understood that it may be possible to dispense with the fastening means 44 in an absorbent article having a given design configuration.

In some embodiments, the fastening means 44 may be adapted to engage or otherwise join with a fastener landing material 45. In some embodiments, the fastener landing material 45 is a loop material joined to the outercover 32 in the front waist region 23 and adapted to engage hook-type fastening means 44. In other embodiments, the outercover 32 may function as the fastener landing material 45 and may be adapted to engage hook-type fastening means 44. In yet other embodiments, the fastener landing material 45 may be a film adapted to engage with tape tab fastening means 44.

The absorbent assembly 34 is positioned between the bodyside liner 30 and the outercover 32 to form the absorbent article 20. The absorbent assembly 34 is generally conformable and capable of absorbing and retaining body exudates. The absorbent assembly 34 comprises an absorbent core 80 and at least one core wrap 84. The absorbent core 80 may be a single, integral piece of material or, alternatively, may comprise a plurality of individual separate pieces of material which are operably assembled together.

The absorbent core 80 may have any of a number of shapes and sizes. The absorbent core 80 may suitably comprise various types of wettable, hydrophilic fibrous materials. Examples of suitable materials include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester and polyamide fibers; and synthetic fibers composed of a nonwettable thermoplastic polymer, such as polypropylene fibers, which have been hydrophilized by appropriate means known to those skilled in the art. The absorbent core 80 may also comprise selected blends of the various types of fibers mentioned above. The absorbent core 80 may include a matrix of hydrophilic fibers, such as a web of cellulosic fibers, mixed with particles of a high-absorbency material such as that commonly known as superabsorbent material.

A “superabsorbent or superabsorbent material” refers to a water-swellable, water-soluble organic or inorganic material capable, under the most favorable conditions, of absorbing at least about 20 times its weight and, more desirably, at least about 30 times its weight in an aqueous solution containing 0.9 weight percent sodium chloride. Organic materials suitable for use as a superabsorbent material in conjunction with the present invention can include natural materials such as agar, pectin, guar gum, and the like; as well as synthetic materials, such as synthetic hydrogel polymers. Such hydrogel polymers include, for example, alkali metal salts of polyacrylic acids, polyacrylamides, polyvinyl alcohol, ethylene maleic anhydride copolymers, polyvinyl ethers, methyl cellulose, carboxymethyl cellulose, hydroxypropylcellulose, polyvinylmorpholinone; and polymers and copolymers of vinyl sulfonic acid, polyacrylates, polyacrylamides, polyvinylpyrridine, and the like. Other suitable polymers include hydrolyzed acrylonitrile grafted starch, acrylic acid grafted starch, and isobutylene maleic anhydride polymers and mixtures thereof. The hydrogel polymers are preferably lightly crosslinked to render the materials substantially water insoluble. Crosslinking may, for example, be accomplished by irradiation or by covalent, ionic, van der Waals, or hydrogen bonding. The superabsorbent materials may be in any form suitable for use in absorbent composites including particles, fibers, flakes, spheres, and the like, and combinations thereof. Such superabsorbents are usually available in particle sizes ranging from about 20 to about 1000 microns. The absorbent core 80 can contain from 0 to 100 percent superabsorbent by weight based upon the total weight of the absorbent core. In various embodiments, the absorbent core 80 may have at least 30 percent, at least 40 percent, at least 50 percent, at least 60 percent, at least 70 percent, at least 80 percent or at least 90 percent superabsorbent material based on the total weight of the absorbent core.

The core wraps of the present invention may be a fibrous nonwoven web made from fine diameter thermoplastic fibers with particular pore sizes and air permeability. By thermoplastic fibers it is meant fibers which are formed from polymers such that the fibers can be bonded to themselves using heat or heat and pressure. While not being limited to the specific method of manufacture, meltblown fibrous nonwoven webs have been found to work particularly well. With respect to polymer selection, polyolefin fibers and especially polypropylene-based polymers have been found to work well. The fibers may be hydrophilic or hydrophobic, though it is desirable that one or more of the resultant core wraps be hydrophilic. As a result, the fibers may be treated to be hydrophilic as by the use of a surfactant treatment.

The core wraps may comprise fibers that are meltblown, spunbond, spunlace, spunbond-meltblown-spunbond, coform, or combinations thereof. The core wraps may have a significant amount of stretchability. For example, the structure of the core wraps may include an operative amount of elastomeric polymer fibers. Furthermore, the fibers utilized in the core wraps may be continuous or discontinous.

The core wraps may comprise a stretchable, durable, hydrophilic, fluid pervious substrate. In some embodiments, the core wraps may comprise a coating including a hydrophilicity boosting amount of nanoparticles, wherein such nanoparticles have a particle size of from 1 to 750 nanometers. Examples of suitable nanoparticles include titanium dioxide, layered clay minerals, alumina oxide, silicates, and combinations thereof. Optionally, a nonionic surfactant can be added to the core wraps to provide additional or enhanced benefits.

In another aspect, the core wraps may be treated with a high-energy surface treatment. This high-energy treatment may occur prior to or concurrent with the hydrophilicity boosting composition coating described above. The high-energy treatment may be any suitable high-energy treatment for increasing the hydrophilicity of the core wrap. Suitable high-energy treatments include, but are not limited to, corona discharge treatment, plasma treatment, UV radiation, ion beam treatment, electron beam treatment and combinations thereof.

The core wraps may additionally or alternatively include materials such as surfactants, ion exchange resin particles, moisturizers, emollients, perfumes, natural fibers, synthetic fibers, fluid modifiers, odor control additives, lotions, viscosity modifiers, anti-adherence agent, pH control agents, and the like, and combinations thereof.

The core wraps may be in the form of films, nonwoven webs, and laminates of two or more substrates or webs. Additionally, the core wraps may be textured, apertured, creped, neck-stretched, heat activated, embossed, and micro-strained.

The absorbent core wraps of the present invention may have wet to dry strength ratios above 0.5 and sometimes 1.0 or higher. In addition, the mean flow pore size, as described in U.S. Pat. No. 5,458,592 to Abuto et al. and issued Oct. 17, 1995, may be about 30 microns or less and less than five percent of the total pores for any given area may be 50 microns or greater. In some embodiments, less than one percent of the total pores for a given area may be 50 microns or greater. In some embodiments, at least 85 percent of the fibers of the core wraps have fiber diameters of 8 microns or less. In other embodiments, at least 95 percent of the fibers may have fiber diameters of 7 microns or less. The absorbent core wraps may have a Frazier air permeability of 200 cubic feet per square foot per minute or greater. The core wraps, while in the dry state, may have respective elongation values at peak load in the machine and cross machine directions of 30 percent or less and 40 percent or less.

Other suitable absorbent cores and core wraps are described in commonly assigned U.S. patent application Ser. No. 11/020842 to Abuto et al., entitled, “Stretchable Absorbent Core and Wrap,” filed Dec. 21, 2004, the entirety of which is incorporated herein by reference where not contradictory. The absorbent core wraps may be manufactured by any suitable means, such as, for example the processes described in U.S. Pat. No. 5,458,592 to Abuto et al. and issued Oct. 17, 1995, which is incorporated herein by reference where not contradictory.

The absorbent article of the present invention may also contain a surge portion to advantageously improve the overall fluid intake rate of the absorbent core. The surge portion is typically less hydrophilic than the absorbent core and is configured to collect and temporarily hold fluid surges. This configuration can also help prevent fluid exudates from pooling and collecting on portions of the absorbent core.

Various woven and nonwoven materials can be used to construct the surge portion. For example, the surge portion may be a layer of a spunbonded or meltblown web of polyolefin fibers or a bonded carded web of natural and synthetic fibers. The surge portion may be a substantially hydrophobic material and, optionally, can be treated with a surfactant or otherwise to impart a desired level of wettability and hydrophilicity. The surge portion may also include other wettable fiber materials such as cotton, rayon, wood pulp, inherently wettable synthetic polymers, hydrophilized or surface treated polymers, and the like. The surge portion may be of any desired shape and configuration.

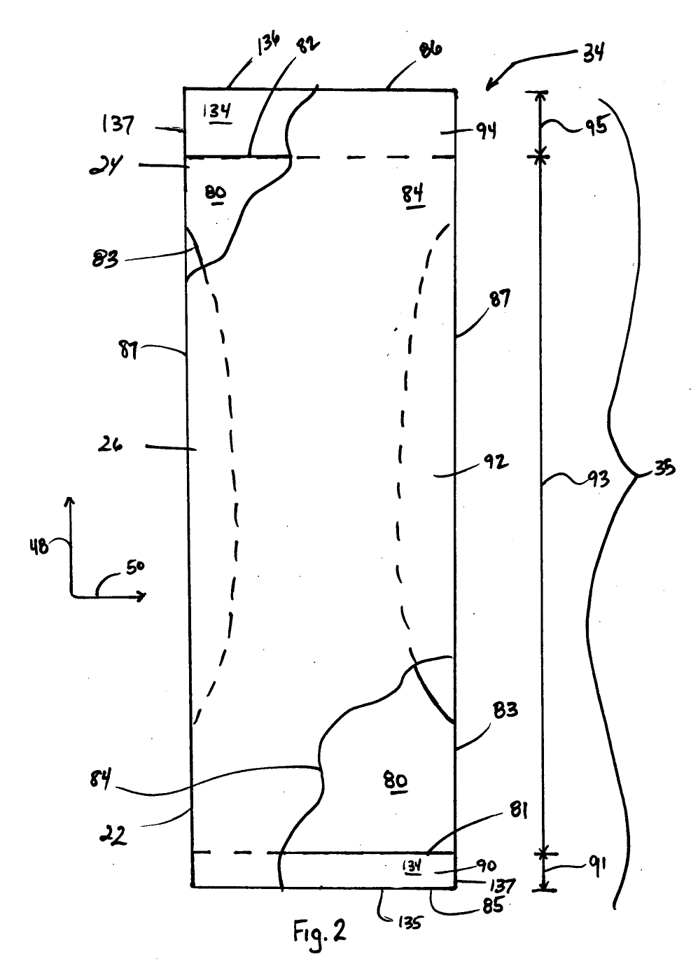

Referring now to FIG. 2, an exemplary absorbent assembly is generally illustrated at 34 with portions cut away to illustrate underlying structure. The absorbent assembly 34 includes an absorbent core 80, a first core wrap 84 and a second core wrap 134.

The absorbent core 80 has a front edge 81 and a rear edge 82. The front edge 81 and the rear edge 82 are generally parallel and are opposed in the longitudinal direction 48. The absorbent core 80 has two side edges 83. The side edges 83 are opposed in the lateral direction 50. The side edges 83 and/or the front edge 81 and/or the rear edge 82 may be straight, arcuate, or other shapes, or combinations thereof. For example, in FIG. 2, the rear edge 82 and the front edge 81 are generally straight, whereas the side edges 83 are generally straight in the front portion 22 and in the rear portion 24 and arcuate in the crotch portion 26.

The first core wrap 84 has a front edge 85 and a rear edge 86. The front edge 85 and the rear edge 86 are generally parallel and are opposed in the longitudinal direction 48. The first core wrap 84 has two side edges 87. The side edges 87 are opposed in the lateral direction 50. The side edges 87 and/or the front edge 85 and/or the rear edge 86 may be straight, arcuate, or other shape, or combinations thereof. For example, in FIG. 2, the rear edge 86, the front edge 85 and the side edges 87 are generally straight.

The second core wrap 134 has a front edge 135 and a rear edge 136. The front edge 135 and the rear edge 136 are generally parallel and are opposed in the longitudinal direction 48. The second core wrap 134 has two side edges 137. The side edges 137 are opposed in the lateral direction 50. The side edges 137 and/or the front edge 135 and/or the rear edge 136 may be straight, arcuate, or other shape, or combinations thereof.

The absorbent assembly 34 has a front extension region 90, a core wrap region 92 and a rear extension region 94. The front extension region 90 has a front extension length 91 as measured in the longitudinal direction 48 from the core wrap front edge 85 to the absorbent core front edge 81. In embodiments including a second core wrap 134, the front extension region 90 is measured using either the front edge 85 of the first core wrap 84 or the front edge 135 of the second core wrap 134 depending on which extends the furthest from the core front edge 81. The core wrap region 92 has a core wrap length 93 as measured in the longitudinal direction 48 from the absorbent core front edge 81 to the absorbent core rear edge 82. The rear extension region 94 has a rear extension length 95 as measured in the longitudinal direction 48 from the absorbent core rear edge 82 to the core wrap rear edge 86. In embodiments including a second core wrap 134, the rear extension region 94 is measured using either the rear edge 86 of the first core wrap 84 or the rear edge 136 of the second core wrap 134 depending on which extends the furthest from the absorbent core rear edge 82. The sum of the front extension length 91, the core wrap length 93 and the rear extension length 95 equals an absorbent assembly length 35.

In various embodiments, the first core wrap 84 may be at least partially bonded to itself, to the second core wrap 134 or both. The first and/or second core wraps may be bonded in the front extension region 90 and/or the core wrap region 92 and/or the rear extension region 94.

In various embodiments, the second core wrap 134 may be at least partially bonded to itself, to the first core wrap 84 or both. The first and/or second core wraps may be bonded in the front extension region 90 and/or the core wrap region 92 and/or the rear extension region 94.

The bonding in the front extension region 90 and/or the rear extension region 94 and/or the core wrap region 92 may be adapted to minimize or eliminate the passage therethrough of absorbent core materials, particularly superabsorbent particles. The bonding in the regions 90, 92 and/or 94 may completely seal the core wrap or wraps thereby preventing any passage of absorbent materials. Alternatively, or additionally, the bonding in the regions 90, 92 and/or 94 may partially seal the core wrap or wraps creating a tortuous path that reduces or eliminates the passage of absorbent materials through the regions 90, 92 and/or 94.

The front extension region 90 and/or the rear extension region 94 may be adapted to minimize or eliminate the passage therethrough of adhesive applied to the outercover 32. The increase in length of the front extension region 90 and/or the rear extension region 94 may be even more beneficial in embodiments including apertures 31 in the bodyside liner 30 to reduce or eliminate adhesive migrating through the apertures 31 and potentially contacting the skin of the wearer.

The first core wrap 84 and/or the second core wrap 134 may be bonded to themselves and/or each other by any suitable means and in any suitable pattern. Suitable bonding means include pressure bonding, thermal bonding, ultrasonic bonding, adhesive bonding, and the like, and combinations thereof. Suitable bonding patterns and techniques are disclosed in commonly assigned U.S. patent application Ser. No. 10/955,769 to Van Himbergen et al. (attorney docket KCC 5009 (K-C 20,724A)), entitled, “Wrapped Absorbent Core,” filed Sep. 30, 2004, the entirety of which is incorporated herein by reference where not contradictory.

In various embodiments, the front extension length 91 may be any suitable length, such as, for example, 1 mm to 150 mm, 6 mm to 50 mm or 15 mm to 30 mm. In various embodiments, the front extension length 91 may be less than 75 mm, less than 50 mm, less than 25 mm or less than 15 mm. In various embodiments, the front extension length 91 may be greater than 1 mm, greater than 5 mm, greater than 10 mm, greater than 15 mm, greater than 20 mm, greater than 25 mm, greater than 30 mm, greater than 40 mm or greater than 50 mm.

In various embodiments, the core wrap length 93 may be any suitable length, such as, for example, 70 mm to 700 mm, 200 mm to 500 mm or 200 mm to 400 mm.

In various embodiments, the core wrap length 93 is less than 95 percent, less than 90 percent, less than 85 percent, less than 80 percent or less than 75 percent of the absorbent assembly length 35.

In various embodiments, the rear extension length 95 may be any suitable length, such as, for example, 1 mm to 150 mm, 50 mm to 125 mm or 75 mm to 125 mm. In various embodiments, the rear extension length 95 may be at least 25 mm, at least 50 mm, at least 75 mm, at least 100 mm, at least 125 mm or at least 150 mm.

In various embodiments, the rear extension length 95 may be at least 5 percent, at least 10 percent, at least 15 percent, at least 20 percent or at least 25 percent of the absorbent assembly length 35.

In various embodiments, the ratio of the rear extension length 95 to the front extension length 91 may be at least 1 to 1, at least 1.5 to 1, at least 2 to 1 or at least 3 to 1. When the ratio of the rear extension length 95 to the front extension length 91 is 1 to 1, the absorbent core 80 is centered within the absorbent assembly 34. When the ratio of the rear extension length 95 to the front extension length 91 is greater than 1 to 1, the absorbent core 80 is skewed towards the front of the absorbent assembly 34. When the ratio of the rear extension length 95 to the front extension length 91 is less than 1 to 1, the absorbent core 80 is skewed towards the rear of the absorbent assembly 34. By altering the position of the absorbent core 80 relative to the absorbent assembly 34, the absorbent assembly 34 can remain centered from front to rear in the absorbent article 20 while allowing the absorbent core 80 to be positioned either towards the front portion 22 or towards the rear portion 24 of the absorbent article 20 as desired.

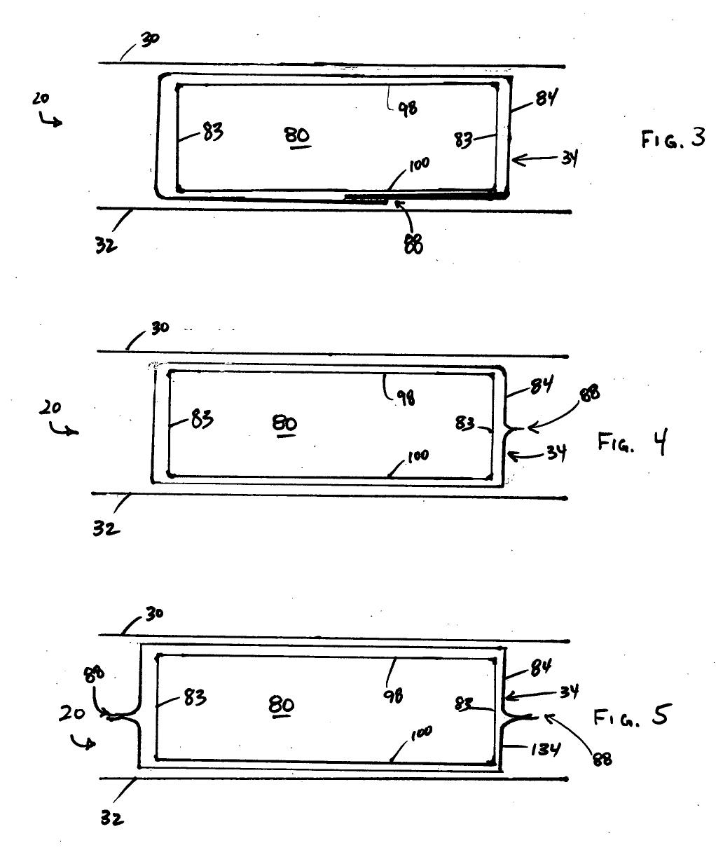

The first core wrap 84 and/or the second core wrap 134 may at least partially envelope the absorbent core 80 in any suitable manner. FIGS. 3-10 representatively illustrate partially exploded cross sectional views of exemplary absorbent articles 20 having absorbent cores 80 at least partially enveloped by at least one core wrap. The absorbent cores 80 have a liner facing surface 98, an outercover facing surface 100 and side edges 83. One skilled in the art will appreciate that many different variations are possible. In various embodiments described herein, the first core wrap 84 and the second core wrap 134 may be interchanged. As used herein, the term “fully envelope” means to enclose or enfold completely within one or more coverings. As used herein, the term “at least partially envelope” means to cover at least one of the liner facing surface 98 and the outercover facing surface 100 of the absorbent cores 80 with one or more coverings.

Referring now to FIG. 3, an absorbent article 20 includes a bodyside liner 30 joined with an outercover 32 and an absorbent assembly 34 located therebetween. The absorbent assembly 34 includes an absorbent core 80 fully enveloped by a core wrap 84. The core wrap 84 is folded around the absorbent core 80 and overlaps itself at a seam 88. In various embodiments, the seam 88 may be located on the liner facing surface 98 or may be located on the outercover facing surface 100, as illustrated in FIG. 3. The seam 88 illustrated in FIG. 3 is a lap seam, alternatively, the seam 88 may be a flange seam, butt seam or any other suitable seam.

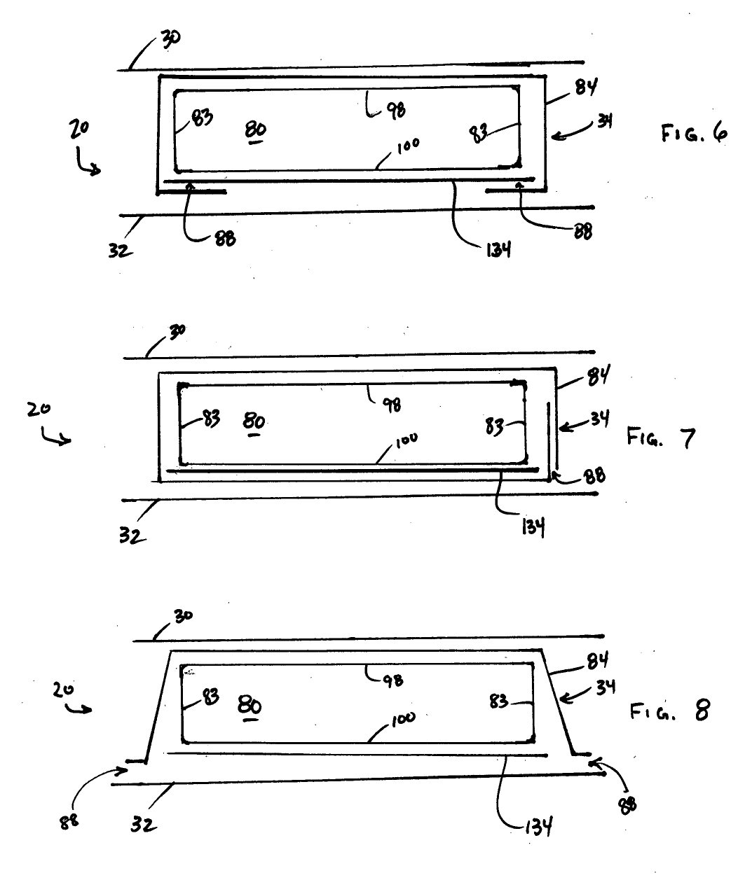

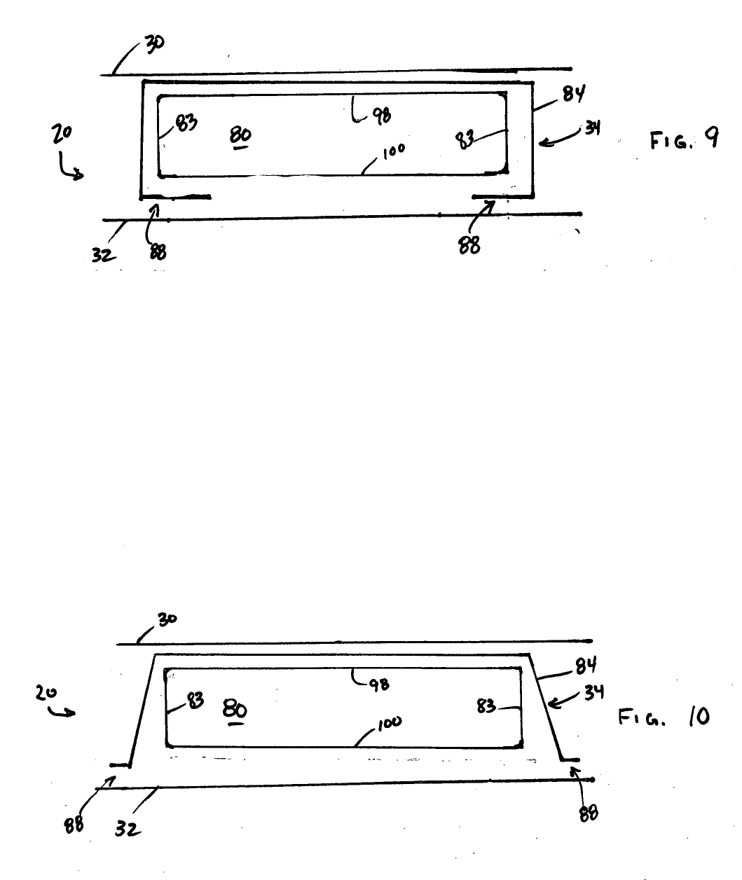

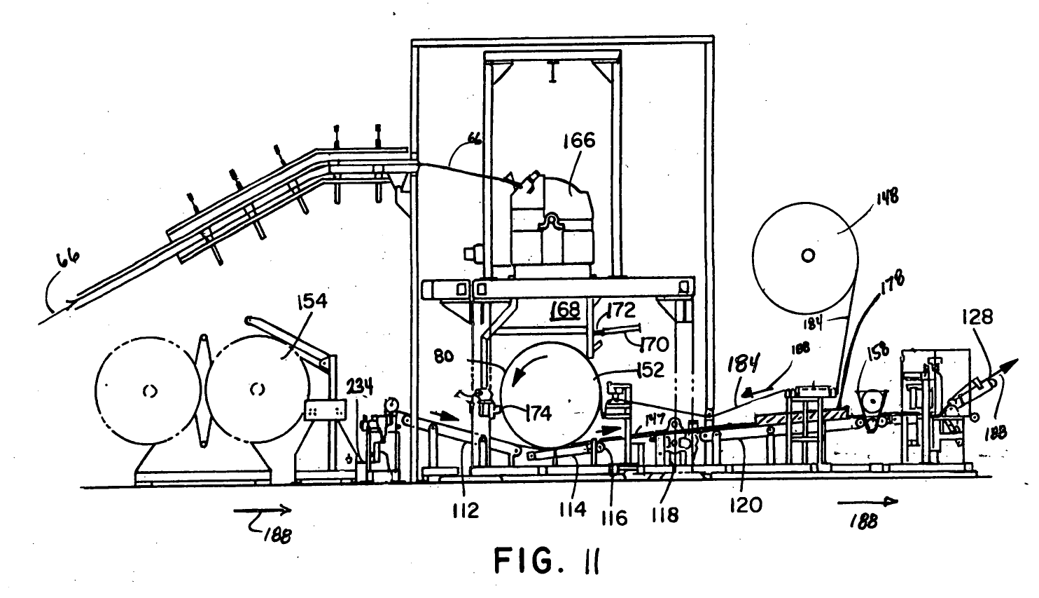

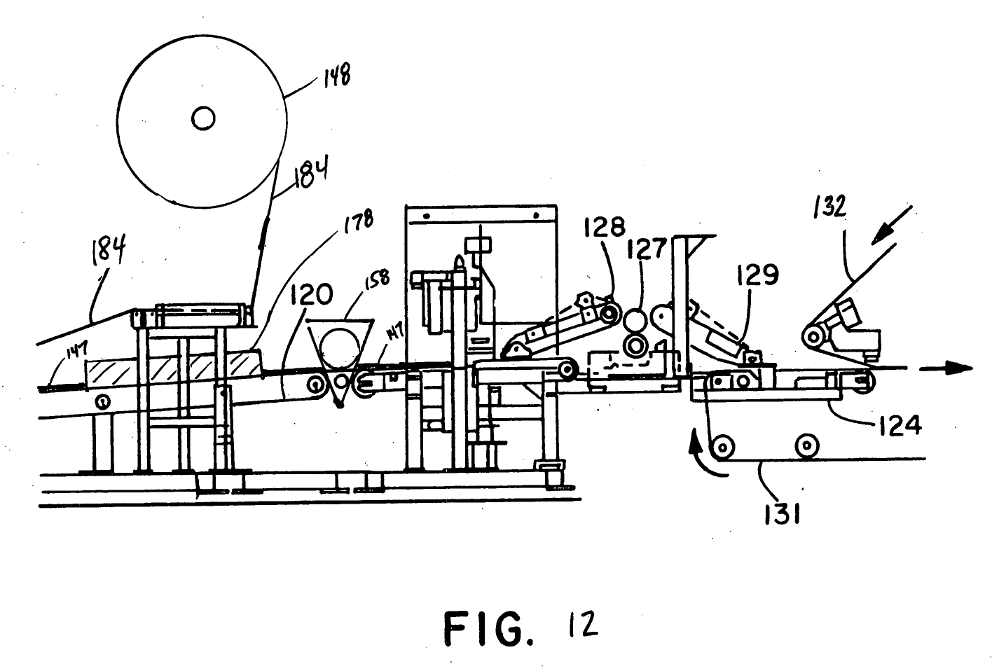

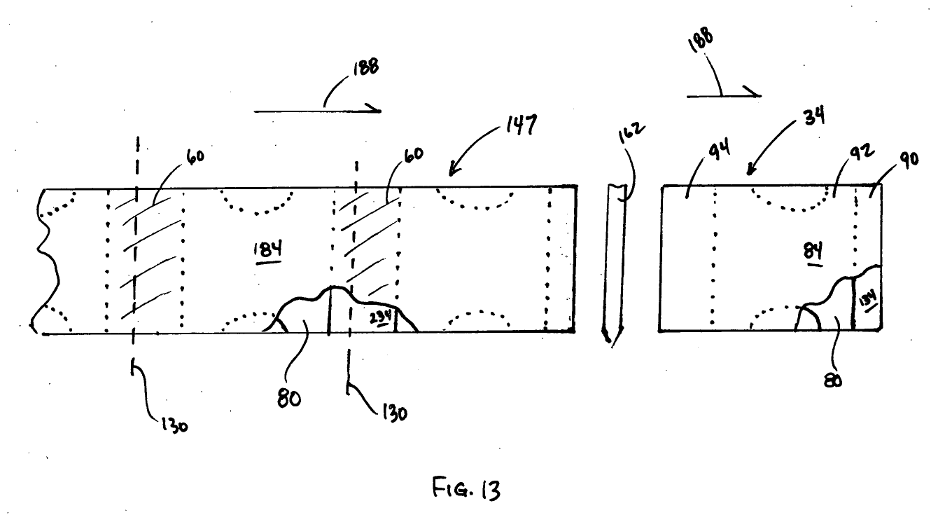

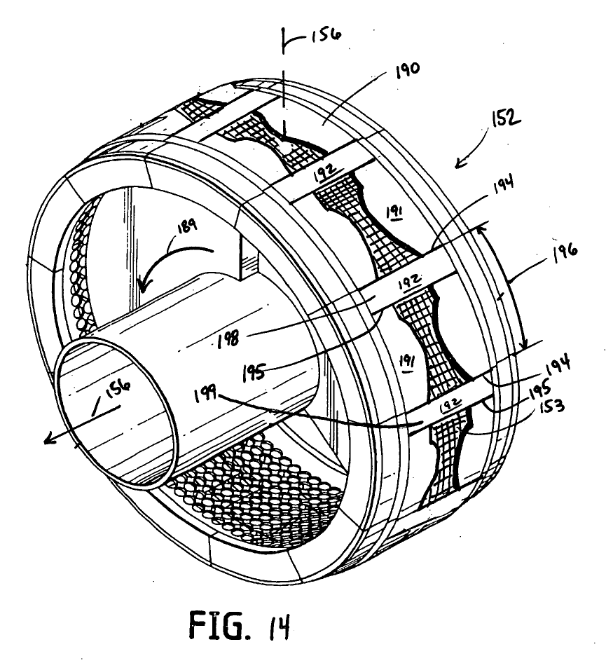

Referring now to FIG. 4, an absorbent article 20 includes a bodyside liner 30 joined with an outercover 32 and an absorbent assembly 34 located therebetween. The absorbent assembly 34 includes an absorbent core 80 fully enveloped by a core wrap 84. The core wrap 84 is folded around the liner facing surface 98, the side edges 83 and the outercover facing surface 100 of the absorbent core 80 and overlaps itself at a seam 88. In various embodiments, the seam 88 may be located on either side edge 83 of the absorbent core 80. The seam 88 illustrated in FIG. 4 is a flange seam, alternatively, the seam 88 may be a lap seam, butt seam or any other suitable seam.

Referring now to FIG. 5, an absorbent article 20 includes a bodyside liner 30 joined with an outercover 32 and an absorbent assembly 34 located therebetween. The absorbent assembly 34 includes an absorbent core 80 fully enveloped by a combination of a first core wrap 84 and a second core wrap 134. The first core wrap 84 is folded around the liner facing surface 98 of the absorbent core 80 and partially around the side edges 83. The second core wrap 134 is folded around the outercover facing surface 100 of the absorbent core 80 and partially around the side edges 83. The first and second core wraps 84 and 134 overlap at seams 88. The seams 88 illustrated in FIG. 5 are flange seams, alternatively, the seams 88 may be lap seams, butt seams, or any other suitable seam. In alternative embodiments, the first core wrap 84 may overlie the outercover facing surface 100 and the second core wrap 134 may overlie the liner facing surface 98.

Referring now to FIG. 6, an absorbent article 20 includes a bodyside liner 30 joined with an outercover 32 and an absorbent assembly 34 located therebetween. The absorbent assembly 34 includes an absorbent core 80 fully enveloped by the combination of a first core wrap 84 and a second core wrap 134. The first core wrap 84 overlies the liner facing surface 98 of the absorbent core 80 and the side edges 83. The second core wrap 134 generally overlies the outercover facing surface 100 of the absorbent core 80 and the first core wrap 84 folds around the absorbent core side edges 83 and overlaps the second core wrap 134 at seams 88. In various embodiments, the second core wrap 134 may overlie the liner facing surface 98 of the absorbent core 80 and the first core wrap 84 may overlie the outercover facing surface 100 of the absorbent core 80. The first core wrap 84 may fold around the absorbent core side edges 83 and overlap the second core wrap 134 at seams 88.

As illustrated, the seams 88 are formed with the first core wrap 84 proximate the outercover 32. In various embodiments, the seams 88 may be formed with the second core wrap 134 proximate the outercover 32. In various embodiments, one of the seams 88 may be formed with the second core wrap 134 proximate the outercover 32 and the other seam 88 may be formed with the first core wrap 84 proximate the outercover 32. The seams 88 illustrated in FIG. 6 are lap seams, alternatively, either or both of the seams 88 may be a flange seam, butt seam or any other suitable seam.

Referring now to FIG. 7, an absorbent article 20 includes a bodyside liner 30 joined with an outercover 32 and an absorbent assembly 34 located therebetween. The absorbent assembly 34 includes an absorbent core 80 fully enveloped by a first core wrap 84. The first core wrap 84 overlies the liner facing surface 98, the side edges 83 and the outercover facing surface 100 and overlaps itself at a seam 88. The absorbent assembly 34 further includes a second core wrap 134 within the first core wrap 84 and overlying the outercover facing surface 100 of the absorbent core 80. In alternative embodiments, the second core wrap 134 may be located between the first core wrap 84 and the outercover 32. The seam 88 illustrated in FIG. 7 is an overlap seam, alternatively, the seam 88 may be a flange seam, butt seam or any other suitable seam.

Referring now to FIG. 8, an absorbent article 20 includes a bodyside liner 30 joined with an outercover 32 and an absorbent assembly 34 located therebetween. The absorbent assembly 34 includes an absorbent core 80 partially enveloped by the combination of a first core wrap 84 and a second core wrap 134. The first core wrap 84 overlies the liner facing surface 98 and the side edges 83 of the absorbent core 80. The first core wrap 84 may be joined with the outercover 32 at seams 88. The second core wrap 134 overlies the outercover facing surface 98 of the absorbent core 80.

Referring now to FIG. 9, an absorbent article 20 includes a bodyside liner 30 joined with an outercover 32 and an absorbent assembly 34 located therebetween. The absorbent assembly 34 includes an absorbent core 80 partially enveloped by a first core wrap 84. The first core wrap 84 overlies the liner facing surface 98 of the absorbent core 80 and the side edges 83. The first core wrap 84 folds around the absorbent core side edges 83 and partially overlies the outercover facing surface 100 of the absorbent core 80 and contacts the outercover at least at seams 88.

Referring now to FIG. 10, an absorbent article 20 includes a bodyside liner 30 joined with an outercover 32 and an absorbent assembly 34 located therebetween. The absorbent assembly 34 includes an absorbent core 80 partially enveloped by a first core wrap 84. The first core wrap 84 overlies the liner facing surface 98 and the side edges 83 of the absorbent core 80 and contacts the outercover 32 at seams 88.

In any of the embodiments described herein, the core wrap and/or wraps and/or outercover may be directly joined together at the seams 88 by any suitable means, such as, for example, by thermal bonding, pressure bonding, ultrasonic bonding, adhesive bonding, and the like, and combinations thereof. In any of the embodiments described herein, the core wrap and/or core wraps and/or outercover may not be directly joined together at the seams 88. In various embodiments, the core wrap and/or core wraps and/or outercover may be directly joined with the absorbent core 80 by thermal bonding, pressure bonding, ultrasonic bonding, adhesive bonding, and the like, and combinations thereof.

In various embodiments, the first core wrap 84 and/or the second core wrap 134 may be a hydrophobic barrier layer. The hydrophobic barrier layer may comprise breathable fibrous materials such as a woven or nonwoven fabric, including but not limited to, meltblown webs, fine fiber spunbond webs such as those having fiber deniers of about 2 or less, bonded and carded webs, hydroentangled fabrics and other fabrics having the similar properties. Suitable polymeric materials for making the barrier layer include those capable of making fibrous webs; examples include but are not limited to polyamides, polyesters and polyolefins, such as polyethylene and/or polypropylenes. In a preferred aspect the hydrophobic barrier layer may comprise a meltblown web of polypropylene fibers having a basis weight from 16 g/m² to about 64 g/m² or from 20 g/m² to 40 g/m². The fibrous barrier layer may comprise a single sheet or multiple layered sheets which collectively have the desired characteristics.

In various embodiments, a first core wrap may have an unfolded width measured in the lateral direction 50. In various embodiments, a second core wrap may have an unfolded width measured in the lateral direction 50. The ratio of the unfolded width of the first core wrap to the unfolded width of the second core wrap may be at least 1 to 1, at least 1.2 to 1, at least 1.5 to 1, at least 1.75 to 1, at least 2 to 1, at least 2.2 to 1, at least 3 to 1, at least 4 to 1, at least 5 to 1 or at least 6 to 1. By folding one or more core wraps about the lateral side edges of the absorbent cores, superabsorbent material can effectively be stopped from migrating out the lateral side edges of the absorbent cores.

As used herein, the terms “first” and “second” are used only for clarity. In any of the embodiments described herein, the first item and the second item may be interchangeable.

A method for making absorbent articles, including those described herein, generally includes the steps of forming discrete absorbent cores on a core wrap web to create a composite web, folding the core wrap web at least partially around the absorbent cores, at least partially bonding the composite web between the absorbent cores and cutting the composite web between the absorbent cores into discrete absorbent assemblies.

As used herein, the term “forming discrete absorbent cores” refers to the process of creating single, unattached absorbent cores wherein a first absorbent core is formed and is not directly connected with a subsequent absorbent core. Forming discrete absorbent cores is in contrast to a process wherein a web of interconnected absorbent cores are formed and then cut apart to form separate cores.

Alternatively, a method for making absorbent articles, including those described herein, may include the steps of forming discrete absorbent cores on a first core wrap web, sandwiching the discrete absorbent cores between the first core wrap web and a second core wrap web to create a composite web, at least partially bonding the composite web between the absorbent cores and cutting the composite web between the absorbent cores into discrete absorbent assemblies. In some embodiments, one or both of the first and second core wrap webs may be folded, at least partially, around the absorbent cores within the composite web. In some embodiments, the first and second core wrap webs may additionally be at least partially bonded along the side edges of the absorbent cores.

The steps of folding, bonding and cutting may be performed in any suitable order and may occur substantially simultaneously and/or may be accomplished with multiple units or with a single unit adapted to perform multiple functions.

The resultant absorbent assemblies may then be located between a bodyside liner and an outercover to form an absorbent article as is known in the art. Other absorbent article components, such as, for example, flaps, elastics, fasteners, and the like, and combinations thereof, may be added as is known in the art and as is described herein.

The method and apparatus of the present invention may be particularly useful when forming fragile or unstable absorbent cores. For example, the absorbent cores formed by the methods and apparatus described herein may be composed of a selected mixture of absorbent hydrophilic fibers and superabsorbent particles. In particular aspects of the invention, the absorbent cores may be constructed to contain at least about 30 percent by weight of superabsorbent material. In some embodiments, the method and apparatus may be used to form absorbent cores containing at least 38 percent, at least 43 percent, at least 50 percent, at least 55 percent, at least 60 percent, at least 65 percent, at least 70 percent or at least 75 percent superabsorbent particles by weight. In some embodiments, the method and apparatus of the present invention may be used to form absorbent cores having more than 75 percent superabsorbent material by weight.

As the weight percentage of superabsorbent increases, the amount of absorbent core integrity resulting from fiber intertwining generally is reduced, thereby resulting in an absorbent core that is more difficult to process and is inherently more fragile. Additionally, low fiber integrity and high superabsorbent concentrations result in more “free” superabsorbent particles capable of moving within and without the absorbent core.

With reference to FIGS. 11 and 12, an exemplary method and apparatus for forming discrete absorbent cores 80 interposed between a first core wrap web 184 and a second core wrap web 234 is illustrated. FIG. 12 representatively illustrates a continuation of the method and apparatus illustrated in FIG. 11. The method and apparatus includes a first web supplying means such as a first supply roll 148 for providing a first core wrap web 184. A depositing means, such as forming drum 152, deposits a series of discrete absorbent cores 80 onto the first core wrap web 184. A second web supplying means, such as a second supply roll 154, provides a second core wrap web 234 to sandwich the discrete absorbent cores 80 between the first core wrap web 184 and the second core wrap web 234 resulting in a composite web 147. The various webs move in a machine direction as indicated by arrows 188.

In alternative embodiments, the method and apparatus may exclude a second core wrap web and may include only a first core wrap web. In yet other alternative embodiments, three or more core wrap webs may be included in the method and apparatus of the present invention. In yet other embodiments, a first core wrap and a second core wrap may be interchanged such that the series of discrete absorbent cores are deposed onto the second core wrap web then joined with the first core wrap web.

A folding apparatus 178 is adapted to fold the first core wrap web 184 and/or the second core wrap web 234 to at least partially envelope the series of discrete absorbent cores 80.

The composite web 147 is transported to a bonding module 158 which at least partially bonds the composite web 147 in attachment regions 60 between the absorbent cores 80 (FIG. 13). The bonding within the attachment regions 60 is adapted to reduce or prevent movement of superabsorbent material from the absorbent cores 80 through the attachment region 60. In general, the greater the percentage of bonding in the attachment region 60, the greater the reduction in superabsorbent movement though the attachment region 60.

A separating means, such as cutting mechanism 127 (FIG. 12) separates the composite web 147 along dividing lines 130 in the attachment regions 60 into discrete absorbent assemblies 34 (FIG. 13).

The shown embodiment of the invention airlays fibrous absorbent material directly onto the first core wrap web 184, and includes a hammermill fiberizer 166 and a rotatable forming drum 152. Fiberizer 166 disintegrates sheets of wood pulp fiber 66 or other suitable fibers and introduces the individual fibers into forming chamber 168. In addition, a superabsorbent supplying means, such as provided by supply conduit 170 and nozzle 172, selectively introduces particles of superabsorbent material into forming chamber 168.

Quantities of superabsorbent material may be continuously introduced into the forming chamber or individual quantities of superabsorbent material may be intermittently introduced into the forming chamber with a pulsing mechanism. The chosen technique will depend upon the desired distribution of superabsorbent across the area and through the thickness of the absorbent cores. Suitable techniques for introducing particles of superabsorbent material into a forming chamber are described in U.S. Pat. No. 6,416,697 issued Jul. 9, 2002 to Venturino et al., and U.S. Pat. No. 5,028,224 issued Jul. 2, 1991 to Pieper et al., the disclosures of which are hereby incorporated by reference where not contradictory.

Referring now to FIG. 14, an exemplary forming drum 152 is illustrated. The forming chamber 168, the first core wrap web 184 and the absorbent cores 80 are not shown to better illustrate the underlying apparatus. The forming drum 152 has a peripheral outer surface 190 and includes a mechanism for forming a vacuum therein to draw the wood pulp fibers and superabsorbent particles onto a core wrap web. The core wrap web is carried by the peripheral outer surface 190 of rotatable forming drum 152, which moves the core wrap web through the forming chamber generally in the direction indicated by arrow 189. The peripheral surface 190 of the forming drum 152 includes an air permeable forming screen 153. As air is drawn though the forming screen 153 by the vacuum within the forming drum 152, wood pulp fibers and superabsorbent particles are drawn onto the first core wrap web to generate a series of discrete airlaid absorbent cores which are substantially regularly spaced along the machine direction length of core wrap web. Suitable techniques of vacuum forming are described in U.S. Pat. No. 6,630,096, issued Oct. 7, 2003 to Venturino et al. and U.S. Pat. No. 6,630,088, issued Oct. 7, 2003 to Venturino et al., the disclosures of which are incorporated herein by reference where not contradictory.

The forming drum 152 includes a plurality of contour rings 191 joined to the peripheral outer surface 190. The contour rings 191 are located about both sides of the forming drum 152 and extend around the circumference of the forming drum 152. The contour rings 191 partially block the forming screens 153 and generally direct the superabsorbent and/or fibers to the unblocked portions of the forming screen 153 thereby forming the absorbent cores 80 and defining the side edges 83 (e.g., FIG. 2).

The forming drum 152 also includes a plurality of cross plates 192 joined to the peripheral outer surface 190, the contour rings 191 or both. The cross plates 192 extend generally perpendicularly between the contour rings 191. The cross plates 192 partially block the forming screen 153 and generally direct the superabsorbent and/or fibers to the unblocked portions of the forming screen 153 thereby forming the absorbent cores 80 and defining the absorbent core front edge 81 and the absorbent core rear edge 82 (FIG. 2).

One advantage of the present apparatus and method is the formation of discrete absorbent cores 80 without cutting the absorbent cores 80. This is accomplished by discretely forming the absorbent cores at pitch.

The plurality of cross plates 192 defines a first edge 194 and a second edge 195. The distance, as measured about the circumference of the forming drum 152, from the first edge 194 of a first cross plate 198 to the first edge 194 of a second sequential cross plate 199, defines a forming pitch 196. The forming pitch 196 equates to the absorbent assembly length 35 (FIG. 2).

The distance, as measured about the circumference of the forming drum 152, from the first edge 194 of a first cross plate 198 to the second edge 195 of the first cross plate 198, defines the spacing between absorbent cores 80. The spacing between absorbent cores 80 equates to the length of the attachment region 60 which in turn equates to the sum of the front extension length 91 and the rear extension length 95.

The first core wrap web 134 overlays at least a portion of the peripheral outer surface 190 of the forming drum 152. Both the forming drum 152 and the first core wrap web 134 move in the direction 189. Vacuum is drawn through the forming screen 153 and first core wrap 134 in the direction indicated by arrows 156 which in turn draws the superabsorbent material and/or fibrous material onto the core wrap web 184. The contour rings 191 and the cross plates 192 substantially block the vacuum in select areas of the forming screens 153 thereby substantially preventing the accumulation of absorbent material on the core wrap web 184 in areas wherein the core wrap web 184 overlies the contour rings 191 and/or cross plates 192. Guiding absorbent material in this way results in discrete cores 80 being formed on the core wrap web 134 in the unblocked portions of the forming screen 153.

Referring again to FIG. 11, a scarfing mechanism is located at an exit end of forming chamber 168. The shown scarfing mechanism includes a scarfing drum 174 which is rotatably driven to operably remove excess absorbent material from the individual absorbent cores 80. The removed material may optionally be recycled back into forming chamber 168. Suitable scarfing methods and apparatus are discussed in U.S. Pat. No. 6,627,130, issued Sep. 30, 2003 to Kugler et al., the disclosure of which is incorporated herein by reference where not contradictory.

Upon leaving the position of scarfing drum 174, the core wrap web 184 and the series of absorbent cores 80 formed thereon may be removed from the forming drum 152 and placed upon a second core wrap web 234. At a transfer screen 114, a second core wrap web 234 may be mated with the series of absorbent cores 80 and the associated first core wrap web 184 coming off from the surface of the forming drum 152 to form the composite web 147. A transfer conveyor 116 may move the composite web 147 from its position near the forming drum 152 into the nip between a pair of debulker rolls 118. The debulker rolls 118 are set and resiliently held at a selected gap, and operably compress together the core wrap web 184, absorbent cores 80 and the second core wrap web 234. This compression densifies the absorbent cores 80 and may at least partially bond the first core wrap web 184 to the second core wrap web 234 in some embodiments.

Upon leaving debulker rolls 118, a debulker conveyor 120 transports the debulked composite web 147 to a folding means such as, for example, folding system 178. Folding system 178 may be adapted to wrap the first core wrap web 184 and/or the second core wrap web 234, if utilized, or both the first core wrap web 184 and the second core wrap web 234 to at least partially envelope the series of discrete absorbent cores 80 within the composite web 147.

Suitable folding systems are described in commonly assigned U.S. patent application Ser. No. 10/955,820 (attorney docket KCC 5009.1 (K-C 20,724B)) to Mischler et al. filed Sep. 30, 2004, and entitled “Method and Apparatus for Making a Wrapped Absorbent Assembly”, the entirety of which is incorporated herein by reference where not contradictory.

After exiting the folding system 178, the composite web 147 may be directed to a bonding module 158 which at least partially bonds the first core wrap web 184 and/or the second core wrap web 234 in an attachment region 60. (FIG. 13).

The illustrated embodiments include a first and a second core wrap web. However, in an alternative embodiment, the method may include forming discrete absorbent cores on a first core wrap web to form a composite web, folding the first core wrap web about the discrete absorbent cores, bonding, debulking and cutting the composite web between the absorbent cores to create discrete absorbent assemblies. In various embodiments, the method steps of folding, bonding and debulking may occur in any order. In various embodiments, the method steps of debulking and bonding may occur in a single module adapted to both debulk the absorbent cores and bond the composite web between the absorbent cores. In various embodiments, the debulking step may be omitted.

In one embodiment, the method may include forming discrete absorbent cores on a first core wrap web, applying adhesive to a second core wrap web, bringing the first and second core wrap webs together in facing relation with the discrete absorbent cores located therebetween to form a composite web. The composite web may be folded, debulked, and bonded in any order. The composite web is then cut between the absorbent cores to form absorbent assemblies. In some embodiments, bonding may be accomplished, at least in part, by pressing the first core wrap web against the second core wrap web in the areas between the absorbent pads to join the webs together utilizing the adhesive previously applied.

In embodiments, wherein the bonding occurs, at least in part, with adhesive, one skilled in the art will recognize that the adhesive may be applied to either the first core wrap web, the second core wrap web or both at any suitable location within the process.

Various types of mechanisms may be employed in the bonding module 158 to form bonds within the attachment regions 60. For example, the attachment regions 60 may include bonds formed by adhesive bonding, thermal bonding, ultrasonic bonding, pressure bonding, or the like, or combinations thereof. Where adhesive bonding is employed, the adhesive may be applied by patterned extrusion, patterned spraying, patterned printing or the like. The patterns may be configured to substantially avoid placing excessive amounts of adhesive onto the fibrous, hydrophilic material used to construct absorbent cores 80.

Exemplary thermal bonding systems are described in U.S. Pat. No. 5,900,109 to Sanders et al. and issued May 4, 1999, the entirety of which is incorporated herein by reference where not contradictory. Exemplary ultrasonic bonding systems are described in U.S. Pat. No. 5,817,199 to Brennecke et al. and issued Oct. 6, 1998, the entirety of which is incorporated herein by reference where not contradictory. Exemplary pressure bonding systems are described in commonly assigned U.S. patent application Ser. No. 11/138099 to Serapati et al., filed May 26, 2005, entitled “Bonding by Induced High-Rate of Shear Deformation”, the entirety of which is incorporated herein by reference where not contradictory. Exemplary adhesive bonding systems are described in U.S. Pat. No. 5,342,647 to Heindel et al. and issued Aug. 30, 1994, the entirety of which is incorporated herein by reference where not contradictory.

The composite web 147 is transported along conveyor 128 to a cutting mechanism 127. The cutting mechanism 127 may be provided by any suitable apparatus, such as, for example, a rotary knife or other suitable cutting means. The cutting mechanism 127 separates the composite web 147 along appointed dividing lines 130 (FIG. 13) to provide individual absorbent assemblies 34. The representatively shown absorbent assemblies 34 include an absorbent core 80 enveloped by a first core wrap 84 and a second core wrap 134.

Referring now to FIG. 13, a top plan view of a portion of the composite web 147 is shown after leaving the bonding module 158. Portions of FIG. 13 have been cut away to illustrate underlying structure. The composite web 147 moves in the machine direction 188. The composite web 147 includes a first nonwoven core wrap web 184 in facing relation with a second nonwoven core wrap web 234. A series of discrete absorbent cores 80 are disposed between the first nonwoven core wrap web 184 and the second nonwoven core wrap web 234. The first core wrap web 184 and the second core wrap web 234 are at least partially bonded together in the attachment regions 60. Cutting mechanism 127 divides the composite web 147 at the dividing lines 130 to create absorbent assemblies 34. The dividing lines 130 are registered to cut between the absorbent cores 80 resulting in each absorbent assembly 34 comprising a front extension region 90 and a rear extension region 94.

The dividing lines 130 may be located at any position between the absorbent cores 80. For example, the dividing lines 130 may be positioned such that attachment region 60 is divided in half in the machine direction 188. Alternatively, the dividing line 130 may be skewed such that attachment region 60 is divided unequally as illustrated in FIG. 13. In other words, the resultant front extension regions 90 and the rear extension regions 94 may be of different lengths.

The absorbent assemblies 34 include a first core wrap 84 and a second core wrap 134 in facing relation. An absorbent core 80 is disposed between the first core wrap 84 and the second core wrap 134. The first core wrap 84 and the second core wrap 134 are at least partially bonded together in the front extension region 90 and the rear extension region 94. The bonding may occur in any suitable pattern or concentration. For example, the bonding may include discrete bond points scattered throughout the front extension region 90 and the rear extension region 94. In another example, the bonding may include adhesive covering essentially the entire front extension region 90 and the entire rear extension region 94. One skilled in the art will appreciate that many different patterns, combinations and coverage areas are possible.

Once divided, the absorbent assemblies 34 may then be transported along a conveyor 129 to a tacker station 124 where the absorbent assemblies 34 are sandwiched and operably attached between a web of bodyside liner 131 and a web of outercover 132. More particularly, the illustrated embodiment of conveyor 129 is configured in a conventional manner to position a series of absorbent assemblies at predetermined, spaced-apart locations along the longitudinal, length dimension of bodyside liner web 131. The outercover web 132 can then be directed by a suitable transporting mechanism to a position overlying both bodyside liner web 131 and absorbent assemblies 34. Accordingly, the absorbent assemblies 34 are interposed between the bodyside liner web 131 and the outercover web 132 to form absorbent articles. In a conventional manner, a suitable attaching means, such as adhesive, bonds or otherwise joins together the constituent components of the absorbent article.

In some embodiments, the absorbent assembly 34 is oriented in the absorbent articles 20 such that absorbent assembly 34 is generally centered between the front waist edge 41 and the rear waist edge 43. The absorbent core 80 may be skewed such that the front extension region 90 is smaller than the rear extension region 94. In other words, the absorbent core 80 may be skewed within the absorbent article 20 without the absorbent assembly 34 being skewed in the absorbent article 20. Without wishing to be bound by theory, it is believed that skewing the absorbent core 80 towards the front portion 22 of the absorbent article 20 may improve the performance of the absorbent article with some users.

Having thus described the invention in detail, it will be readily apparent to a person of ordinary skill that various changes and modifications can be made without departing from the spirit of the invention. All of such changes and modifications are contemplated as being within the scope of t he present invention.

1. An absorbent article comprising,
a. an outercover,
b. a bodyside liner, and
c. an absorbent assembly positioned between the outercover and the bodyside liner, the absorbent assembly comprising, a nonwoven core wrap at least partially enveloping an absorbent core, the absorbent assembly having a rear extension region length of at least 30 mm.

2. The absorbent article of claim 1 wherein the absorbent assembly has a core wrap region length that is less than 90 percent of an absorbent assembly length.

3. The absorbent article of claim 1 wherein the absorbent assembly has a front extension region length and the ratio of the rear extension region length to the front extension region length is at least 1.5 to 1.

4. The absorbent article of claim 1 wherein the absorbent assembly has a front extension region length and the ratio of the rear extension region length to the front extension region length is at least 2 to 1.

5. The absorbent article of claim 1 wherein the nonwoven core wrap comprises thermoplastic fibers.

6. The absorbent article of claim 5 wherein the nonwoven core wrap is folded around the absorbent core and overlaps onto itself.

7. The absorbent article of claim 6 wherein the absorbent assembly has a core wrap region and the nonwoven core wrap is at least partially bonded to itself in the core wrap region.

8. The absorbent article of claim 6 wherein the nonwoven core wrap is at least partially bonded to itself in a front extension region and a rear extension region.

9. The absorbent article of claim 8 wherein the absorbent core comprises at least 60 percent superabsorbent by weight.

10. The absorbent article of claim 1 wherein the absorbent core has a liner facing surface and an outercover facing surface, the nonwoven core wrap overlies the liner facing surface and partially overlies the outercover facing surface, the nonwoven core wrap being adhesively joined with the outercover in a front extension region and the rear extension region.

11. An absorbent article comprising,
a. an outercover,
b. a bodyside liner, and
c. an absorbent assembly positioned between the outercover and the bodyside liner, the absorbent assembly comprising, an absorbent core positioned between a first nonwoven core wrap and a second nonwoven core wrap, the absorbent assembly having a rear extension region length of at least 30 mm.

12. The absorbent article of claim 11 wherein the absorbent core comprises at least 60 percent superabsorbent material by weight.

13. The absorbent article of claim 12 wherein the bodyside liner comprises a plurality of apertures.

14. The absorbent article of claim 12 wherein the first nonwoven core wrap and the second nonwoven core wrap comprise thermoplastic fibers.

15. The absorbent article of claim 14 wherein the first and the second nonwoven core wraps are at least partially bonded together in at least a front extension region and a rear extension region.

16. The absorbent article of claim 15 wherein the front extension region has a front extension region length and the rear extension region has a rear extension region length and the ratio of the rear extension region length to the front extension region length is at least 2 to 1.

17. The absorbent article of claim 15 wherein the first nonwoven core wrap is hydrophilic and the second nonwoven core wrap is hydrophobic.

18. The absorbent article of claim 11 wherein the absorbent article has a front extension region length of at least 10 mm.

19. An absorbent article comprising,
a. an outercover,
b. a bodyside liner, and
c. an absorbent assembly positioned between the outercover and the bodyside liner, the absorbent assembly comprising an absorbent core positioned between a first thermoplastic hydrophilic nonwoven core wrap and a second thermoplastic hydrophobic nonwoven core wrap, the absorbent core comprising at least 60 percent superabsorbent and the absorbent assembly having a rear extension region length of at least 30 mm.

20. The absorbent article of claim 19 wherein the absorbent core has a liner facing surface, an outercover facing surface and lateral side edges, the first nonwoven core wrap overlies the liner facing surface, the lateral side edges and least a portion of the outercover facing surface, the second nonwoven core wrap at least partially overlies the outercover facing surface, the first nonwoven core wrap being at least partially adhesively bonded to the second nonwoven core wrap in a front extension region and a rear extension region.
